(19) United States
(12) Patent Application Publication
(10) Pub. No.: US 2011/0152640 A1
(43) Pub. Date: Jun. 23, 2011

(54) METHOD AND SYSTEM FOR UTILIZING HEALTH CARE PRODUCTS

(75) Inventor: Michael Stanley Whittingham, Los Gatos, CA (US)

(73) Assignee: SENSORDERM

(21) Appl. No.: 12/640,539

(22) Filed: Dec. 17, 2009

(51) Int. Cl. A61M 35/00; A61B 5/00

(52) U.S. Cl. 600/306; 604/290; 600/300

(57) ABSTRACT

In varying embodiments, a method and system of utilizing health care products is disclosed. The method and system employ a uniquely configured device for measuring a biometric component of the human body. Based on this measured biometric component, health care products can be selectively used that are best suited for the measured biometric component. Consequently, health care products can be utilized in a more precise and accurate fashion. An aspect of the invention is a method for utilizing health care products. The method comprises using a sensory device to measure a biometric component and applying one of a plurality of health care products based on the measured biometric component.

### FIELD OF THE INVENTION

The present invention relates generally to health care products and more particularly to a method and system for utilizing health care products.

### BACKGROUND OF THE INVENTION

The pH (power of hydrogen) factor is a term most of us have heard but which few understand the significance of as it relates to skin care. Calculated on a scale of one to 14, pH refers to the levels of acid or alkaline in a substance. Below seven is acidic, above is alkaline, and seven—the approximate optimum level for the body—is neutral. However, skin has a different pH and to avoid and treat dry skin, it is important to choose natural skin care treatment products that have the proper pH balance.

The pH of normal skin ranges from 4 to 6.5, which is slightly acidic. This acidic environment is referred to as the skin's ‘acid mantle’. It contains a number of different acids including lactic acid, amino acids and free fatty acids. One of the major functions of the acid mantle is to protect the skin and body from the absorption of bacteria. As bacteria cannot survive in an acidic environment, maintaining the correct level of acidity is vital.

Because pH levels vary from person to person, existing skin creams, soaps and makeup do not address each user's specific skin pH issues. Rather manufacturers mass-produce products that claim to help with general pH imbalances when they ought to be focusing on specificity instead. The few existing pH measurement devices that are adaptable to measuring skin are very expensive and difficult to use and therefore are owned and operated by health care professionals.

Consequently, what is needed is a simple and cost effective way to measure pH levels. The present invention addresses this need.

### SUMMARY OF INVENTION

In varying embodiments, a method and system of utilizing health care products is disclosed. The method and system employ a uniquely configured device for measuring a biometric component of the human body. Based on this measured biometric component, health care products can be selectively used that are best suited for the measured biometric component. Consequently, health care products can be utilized in a more precise and accurate fashion.

An aspect of the invention is a method for utilizing health care products. The method comprises using a sensory device to measure a biometric component and applying one of a plurality of health care products based on the measured biometric component.

Another aspect of the invention is a system for utilizing health care products. The system comprises a sensory device for measuring a biometric component, a plurality of health care products and a symbolic schema for associating the measured biometric component with each of the plurality of health care products.

Other aspects and advantages of the present invention will become apparent from the following detailed description, taken in conjunction with the accompanying drawings, illustrating by way of example the principles of the invention.

### BRIEF DESCRIPTION OF THE DRAWINGS

FIG. 1 shows a high level flowchart of a method in accordance with an embodiment of the present invention.

FIG. 2 shows a health care system in accordance with an embodiment of the present invention.

### DETAILED DESCRIPTION OF THE PRESENT INVENTION

A method and system for utilizing health care products is disclosed. The following description is presented to enable one of ordinary skill in the art to make and use the invention and is provided in the context of a patent application and its requirements. Various modifications to the embodiments and the generic principles and features described herein will be readily apparent to those skilled in the art. Thus, the present invention is not intended to be limited to the embodiment shown but is to be accorded the widest scope consistent with the principles and features described herein.

In varying embodiments, a method of utilizing health care products is disclosed. The method employs a uniquely configured device for measuring a biometric component of the human body. Based on this measured biometric component, health care products can be selectively used that are best suited for the measured biometric component. Consequently, health care products can be utilized in a more precise and accurate fashion.

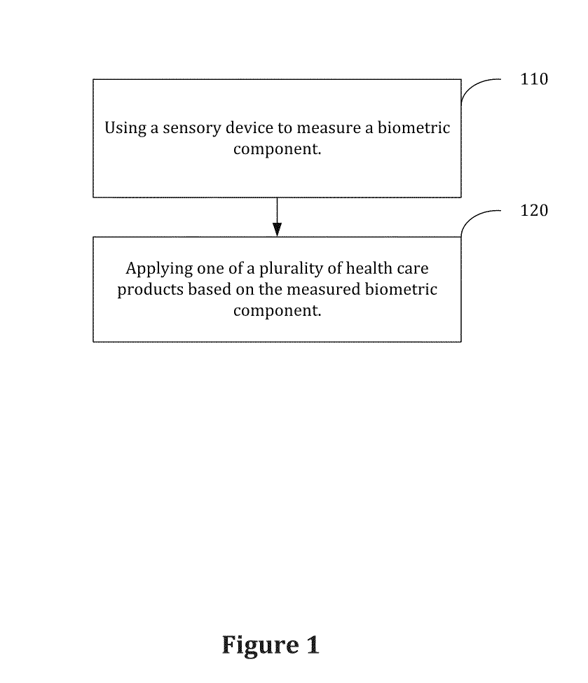

FIG. 1 shows a flowchart of a method in accordance with an embodiment of the present invention. Step 110 comprises using a sensory device to measure a biometric component. Step 120 comprises applying one of a plurality of health care products based on the measured biometric component.

In an embodiment, the biometric component is the pH level of human skin. However, one of ordinary skill in the art will readily recognize that a variety of different biometric components could be measured while remaining within the spirit and scope of the present invention. Additionally, health care products could be skin creams, make up, cosmetics, soaps, shampoos or any of a variety of products while remaining within the spirit and scope of the present invention.

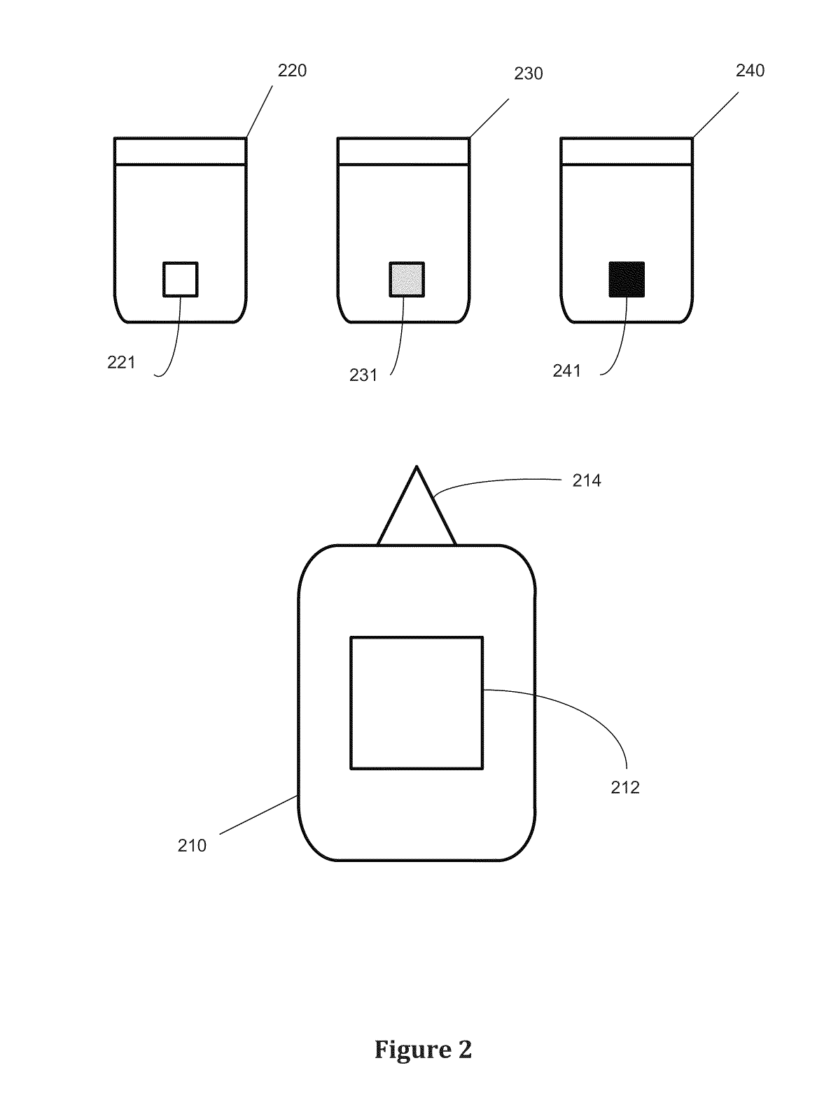

The sensory device employed in conjunction with an embodiment of the present invention, allows the user to quickly and accurately measure the pH level of their skin and/or hair. FIG. 2 shows a health care system 200 in accordance with an embodiment. The health care system 200 includes a sensory device 210 and a plurality of health care products 220-240. The sensory device 210 includes a user interface 212 and a biometric measurement mechanism 214. Once the pH level is measured, a symbolic schema is then employed to associate the measured pH level with one of the plurality of health care products.

For instance, when a user measures his or her skin pH with the sensory device 210, the user interface 212 displays the measured pH value in the form of one of a plurality of symbols wherein each of the plurality of symbols is representative of a specific measured pH level. For example, if the symbols are different colors, each color can represent a pH level or range of pH readings e.g. red can be assigned to pH levels between 3.0 and 4.0, blue to levels between 4.0 and 5.0 green to levels 5.0 to 6.0, etc. Here, each of the plurality of health care products 220-240 is associated with a particular color 221, 231, 241. Accordingly, when a user touches their skin with the biometric measurement mechanism 214, the user interface 212 will display one of the colors. Consequently, the user will select the one of the plurality of health care products 220-240 that corresponds with the displayed color.

Although the above described embodiment is described in the context of being utilized in conjunction with a color schema, one of ordinary skill in the art will readily recognize that a variety of different schema could be employed. For example, a set of distinct symbols (triangle, square, etc.) could be used to differentiate the different health care products.

Since pH levels are prone to change from day to day, the system 200 removes any guesswork the consumer may have with regard to the use of the health care products. In fact, consumers don't even need to know that pH is being measured, only that the system 200 helps them choose health care products specifically suited to their unique pH measurements.

Additionally, by using the disclosed symbolic schema, the sensory device can be “locked” to a particular set of products. More specifically, the color coding can be customized based on any set of predetermined criteria. Accordingly, products can be manufactured that have the matching schema or the schema can be shared with a specific product manufacturer(s) whereby no other manufacturer would be capable of creating a compatible product. Thus, the user interface would enable a device that would only work with specific health care products.

A method of utilizing health care products is disclosed. The method employs a uniquely configured device for measuring a biometric component of the human body. Based on this measured biometric component, health care products can be selectively used that are best suited for the measured biometric component. Consequently, health care products can be utilized in a more precise and accurate fashion.

Without further analysis, the foregoing so fully reveals the gist of the present inventive concepts that others can, by applying current knowledge, readily adapt it for various applications without omitting features that, from the standpoint of prior art, fairly constitute essential characteristics of the generic or specific aspects of this invention. Therefore, such applications should and are intended to be comprehended within the meaning and range of equivalents of the following claims. Although this invention has been described in terms of certain embodiments, other embodiments that are apparent to those of ordinary skill in the art are also within the scope of this invention, as defined in the claims that follow.

What is claimed is:

1. A method for utilizing health care products comprising:
using a sensory device to measure a biometric component;
applying one of a plurality of health care products based on the measured biometric component.

2. The method of claim 1 wherein applying one of a plurality of health care products further comprises employing a symbolic schema to associate the measured biometric component with one of the plurality of health care products.

3. The method of claim 2 wherein the symbolic schema associates a plurality of indicators with a plurality of measured biometric components.

4. The method of claim 3 wherein each of the plurality of indicators is a distinct color.

5. The method of claim 3 wherein each of the plurality of indicators is a distinct symbol.

6. The method of claim 3 wherein the biometric component comprises a pH level of skin.

7. The method of claim 3 wherein the biometric component comprises a pH level of hair.

8. The method of claim 3 wherein the plurality of health care products comprise at least one of creams, soaps, cosmetics and shampoos.

9. A system for utilizing health care products comprising:
a sensory device for measuring a biometric component;
a plurality of health care products; and
a symbolic schema for associating the measured biometric component with each of the plurality of health care products.

10. The system of claim 9 wherein the symbolic schema associates a plurality of indicators with a plurality of measured biometric components.

11. The system of claim 10 wherein each of the plurality of indicators is a distinct color.

12. The system of claim 10 wherein each of the plurality of indicators is a distinct symbol.

13. The system of claim 10 wherein the biometric component comprises a pH level of skin.

14. The system of claim 10 wherein the biometric component comprises a pH level of hair.

15. The system of claim 10 wherein the plurality of health care products comprise at least one of creams, soaps, cosmetics and shampoos.
